(19) United States
(12) Patent Application Publication
(10) Pub. No.: US 2007/0225277 A1
(43) Pub. Date: Sep. 27, 2007

(54) Treatment of pain

(75) Inventor: Sharon Rosenzweig-Lipson, East Brunswick, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/726,850

(22) Filed: Mar. 23, 2007

Related U.S. Application Data
(60) Provisional application No. 60/785,633, filed on Mar. 24, 2006.

(51) Int. Cl. A61K 31/551; A61K 31/498

(52) U.S. Cl. 514/220; 514/250

(57) ABSTRACT

This invention provides a method of treating pain in a mammal that includes administering to a mammal in need of such treatment a pain treating effective amount of a compound of the formula I: 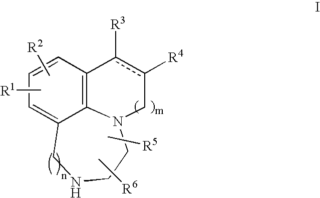
or a pharmaceutically acceptable salt thereof, wherein each of R¹, R², R³, R⁴, R⁵, R⁶, n, and m is as defined and described herein. The present invention also provides pharmaceutical compositions for treating pain containing a pain treating effective amount of a compound of formula I.

### CROSS-REFERENCE TO RELATED APPLICATIONS

The present application claims priority to U.S. provisional patent application Ser. No. 60/785,633, filed Mar. 24, 2006, the entirety of which is hereby incorporated herein by reference.

### BACKGROUND OF THE INVENTION

Pain has been characterized and described in various different ways in the literature. For example, pain can be intense, localized, sharp or stinging, and/or dull, aching, diffuse or burning in nature. Pain can also be centralized (i.e., taking place in the dorsal horn of the spinal cord, the brain stem and brain), or peripheral (i.e., taking place at the injury site and surrounding tissue). Pain that occurs for extended periods of time (i.e., is persistent) is generally referred to as chronic pain. Examples of chronic pain include neuropathic pain, inflammatory pain, and cancer pain. These pains can be related to hyperalgesia and/or allodynia, where hyperalgesia refers to an increase in sensitivity to a typically noxious stimulus and allodynia refers to an increase in sensitivity to a typically non-noxious stimulus.

A type of chronic pain that currently lacks adequate pharmacological treatment is neuropathic pain. Neuropathic pain is generally thought of as a chronic pain caused by damage to or pathological changes in the peripheral or central nervous systems. Examples of pathological changes related to neuropathic pain include prolonged peripheral or central neuronal sensitization, central sensitization related damage to nervous system inhibitory and/or excitatory functions and abnormal interactions between the parasympathetic and sympathetic nervous systems. A wide range of clinical conditions may be associated with or form the basis for neuropathic pain including for example diabetes, post traumatic pain of amputation, lower back pain, cancer, chemical injury or toxins, other major surgeries, peripheral nerve damage due to traumatic injury compression, nutritional deficiencies, or infections such as shingles or HIV.

There are various types of agents currently being used to treat pain such as for example, non-narcotic analgesics such as aspirin, acetaminophen or ibuprofen; non-steroidal anti-inflammatory drugs (NSAIDs); narcotic analgesics such as morphine, hydromorphone, fentanyl, codeine or meperidine; steroids such as prednisone or dexamethasone; tricyclic antidepressants such as amitriptyline, desipramine, or imipramine; antiepileptics such as gabapentin, carbamazepine, topiramate, sodium valproate or phenyloin; or combinations of these different agents. However, these agents are typically unsatisfactory for treating pain of a chronic nature, and can have adverse effects such as drowsiness, dizziness, dry mouth, weight gain, memory impairment, and/or orthostatic hypotension.

There has also been recent interest in treating pain with inhibitors of the N-methyl-D-aspartate (“NMDA”) receptors to treat pain (hereinafter called “NMDA receptor antagonists”). While some such compounds show promise, their clinical usefulness has been limited due to adverse effects such as headache, increased heart rate, increased blood pressure; disturbances of motor function such as ataxia, or sedation; and/or psychotomimetic effects such as dizziness, hallucinations, dysphoria, or disturbances of cognitive function that are observed when they are administered at analgesic doses.

Thus, there remains a need for improved therapies for the treatment of pain.

### SUMMARY OF THE INVENTION

The present invention provides a method of treating pain in a mammal that includes administering to a mammal in need of such treatment a pain treating effective amount of at least one compound having the formula I: 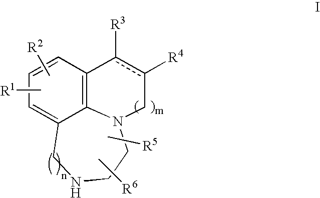
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R.

The present invention also provides pharmaceutical compositions that contain a pain treating effective amount of a compound of formula I, or a pharmaceutically acceptable salt thereof; and at least one pharmaceutical carrier or other ingredient.

In some embodiments of the invention, a compound of formula I is administered in combination with another pain relieving agent and/or with one or more agents for reducing side effects of the pain relieving agent(s).

The present invention also provides pharmaceutical compositions comprising one or more compounds of formula I formulated for administration to treat pain in a mammal. In some embodiments, the pharmaceutical composition is provided in unit dosage form. The present invention further provides therapeutic packages containing one or more compounds of formula I in unit dosage form for treating pain in a mammal.

It is well known that the neurotransmitter 5-HT plays a major role in inhibition of nociceptive transmission. Various studies have demonstrated that at least 4 families of 5-HT receptors are present in pain processing pathways and include 5-HT1, 5-HT2, 5-HT3, and 5-HT4 (1-2). Furthermore, although exact mechanisms are poorly understood, it appears that 5-HT2C receptors have an inhibitory role in neuropathic pain (3-5). In summary, 5-HT2C agonists may be effective in the treatment of diabetic neuropathy, post herpetic neuralgia, low back pain, phantom limb pain, visceral pain (chronic and acute), irritable bowel syndrome pain, irritable bowel disease pain, fibromyalgia and complex regional pain syndrome.

### BRIEF DESCRIPTION OF THE DRAWING

FIG. 1 shows the effectiveness of Compound 1 in the tactile allodynia model.

FIG. 2 shows the effectiveness of Compound 2 in the mechanical hyperalgesia model.

### DETAILED DESCRIPTION OF CERTAIN EMBODIMENTS OF THE INVENTION

1. Compounds

The present invention utilizes 5-HT_{2C} receptor agonists, or partial agonists, of formula I: 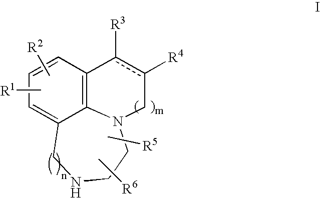
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R.

As used herein, the term “alkyl” includes, but is not limited to, straight and branched chains such as methyl, ethyl, n-propyl, isopropyl, n-butyl, isobutyl, sec-butyl, or t-butyl.

The terms “halogen” or “halo,” as used herein, refer to chlorine, bromine, fluorine or iodine.

The term “perfluoroalkyl,” as used herein refers to an alkyl group, as defined herein, wherein every hydrogen atom on said alkyl group is replaced by a fluorine atom. Such perfluoroalkyl groups include —CF₃.

The terms “effective amount” and “therapeutically effective amount,” as used herein, refer to the amount of a compound or combination that, when administered to an individual, is effective to treat, prevent, delay, or reduce the severity of a condition from which the patient is suffering. In particular, a therapeutically effective amount in accordance with the present invention is an amount sufficient to treat, prevent, delay onset of, or otherwise ameliorate at least one symptom of a psychotic disorder or episode.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

The term “patient,” as used herein, refers to a mammal. In certain embodiments, the term “patient” refers to a human.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

Compounds of formula I, as defined above or in classes and subclasses as described herein, have affinity for and agonist or partial agonist activity at the 2C subtype of brain serotonin receptors.

2. Description of Exemplary Compounds:

In certain embodiments, [symbol] designates a single bond. In other embodiments, [symbol] designates a double bond.

In certain embodiments, the R¹ group of formula I is R, OR, halogen, cyano, or —C₁₋₃ perfluoroalkyl. In other embodiments, the R¹ group of formula I is hydrogen, halogen, cyano, —OR wherein R is C₁₋₃ alkyl, or trifluoromethyl. According to another embodiment, the R¹ group of formula I is hydrogen.

In certain embodiments, the R² group of formula I is R, OR, halogen, cyano, or —C₁₋₃ perfluoroalkyl. In other embodiments, the R² group of formula I is hydrogen, halogen, cyano, —OR wherein R is hydrogen, C₁₋₃ alkyl, or trifluoromethyl. According to another embodiment, the R² group of formula I is hydrogen.

According to one aspect of the present invention, at least one of R¹ and R² groups of formula I is —OH. According to another aspect of the present invention, both of the R¹ and R² groups of formula I are —OH.

According to another embodiment, each of the R¹ and R² groups of formula I is hydrogen. According to yet another embodiment, each of the R⁵ and R⁶ groups of formula I is hydrogen.

As defined generally above, the R³ and R⁴ groups of formula I are taken together to form a saturated or unsaturated 4-8 membered ring, wherein said ring is optionally substituted with 1-3 groups independently selected from halogen, —R, or OR. According to one embodiment, the R³ and R⁴ groups of formula I are taken together to form a saturated or unsaturated 5-8 membered ring, wherein said ring is optionally substituted with 1-3 groups independently selected from halogen, —R, or OR. In certain embodiments, the R³ and R⁴ groups of formula I are taken together to form a saturated or unsaturated 5-6 membered ring, wherein said ring is optionally substituted with 1-3 groups independently selected from halogen, —R, or OR.

As defined generally above, n is 1 or 2. Accordingly, the present invention provides a compound of formulae I-a and I-b: 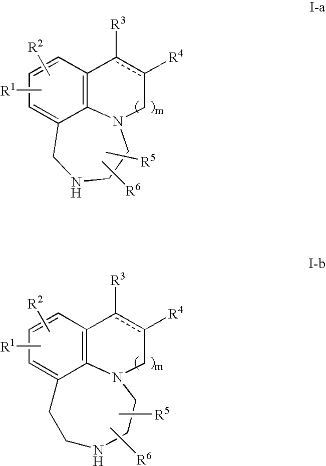
or a pharmaceutically acceptable salt thereof, wherein each of m, R¹, R², R³, R⁴, R⁵, and R⁶ is as defined above for compounds of formula I and described in classes and subclasses above and herein.

As defined generally above, m is 0 or 1. Accordingly, the present invention provides a compound of formulae I-c and I-d: 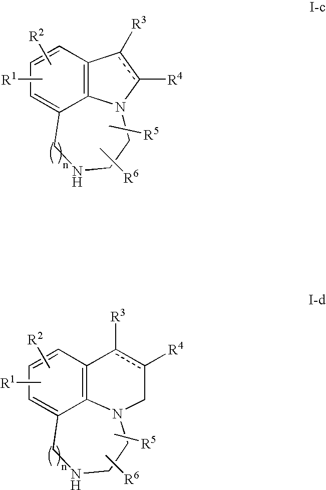
or a pharmaceutically acceptable salt thereof, wherein each of n, R¹, R², R³, R⁴, R⁵, and R⁶ is as defined above for compounds of formula I and described in classes and subclasses above and herein.

In other embodiments, n is 1, m is 1, and the R³ and R⁴ groups of formula I are taken together to form a saturated 5-membered ring and said compound is of formula II: 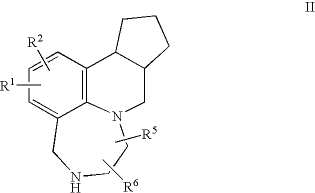
or a pharmaceutically acceptable salt thereof, wherein each of R¹, R², R⁵, and R⁶ is as defined above for compounds of formula I and described in classes and subclasses above and herein.

According to another aspect of the present invention, a compound is provided, wherein n is 1, m is 0, and the R³ and R⁴ groups of formula I are taken together to form a saturated 5-membered ring and said compound is of formula III: 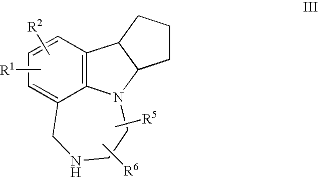
or a pharmaceutically acceptable salt thereof, wherein each of R¹, R², R⁵, and R⁶ is as defined above for compounds of formula I and described in classes and subclasses above and herein.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers.

According to another aspect, the present invention provides a compound of either of formulae I-e or I-f: 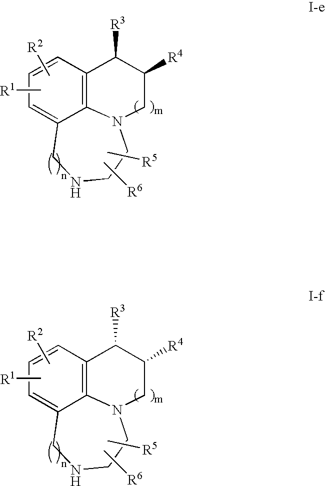
or a pharmaceutically acceptable salt thereof, wherein each of n, m, R¹, R², R³, R⁴, R⁵, and R⁶ is as defined above for compounds of formula I and described in classes and subclasses above and herein.

In certain embodiments, the present invention provides a compound of either of formulae IV or V: 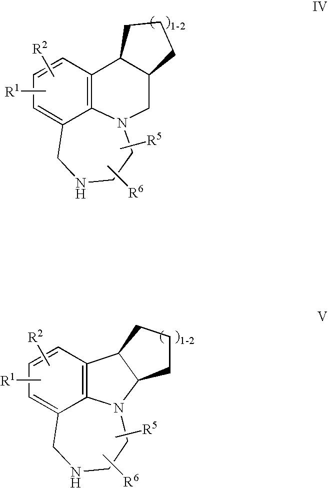
or a pharmaceutically acceptable salt thereof, wherein each R¹, R², R⁵, and R⁶ are as defined above for compounds of formula I and in classes and subclasses as described above and herein.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); Wilen, S. H. Tables of Resolving Agents and Optical Resolutions p. 268 (E. L. Eliel, Ed., Univ. of Notre Dame Press, Notre Dame, Ind. 1972).

Exemplary compounds useful for methods of the present invention are set for the in Table 1, below,

TABLE 1
Exemplary Compounds of Formula I

2-bromo-4,5,6,7,9,9a,10,11,12,12a-decahydrocyclopenta[c][1,4]diazepino[6,7,1-ij]quinoline;
2-bromo-4,5,6,7,9,9a,10,11,12,13,14,14a-dodecahydrocyclohepta[c][1,4]diazepino[6,7,1-
ij]quinoline;
2-chloro-4,5,6,7,9,9a,10,11,12,12a-decahydrocyclopenta[c][1,4]diazepino[6,7,1-ij]quinoline;
2-chloro-4,5,6,7,9,9a,10,11,12,13,14,14a-dodecahydrocyclohepta[c][1,4]diazepino[6,7,1-
ij]quinoline;
2-phenyl-4,5,6,7,9,9a,10,11,12,12a-decahydrocyclopenta[c][1,4]diazepino[6,7,1-ij]quinoline;
2-methoxy-4,5,6,7,9,9a,10,11,12,12a-decahydrocyclopenta[c][1,4]diazepino[6,7,1-ij]quinoline;
1-fluoro-4,5,6,7,9,9a,10,11,12,12a-decahydrocyclopenta[c][1,4]diazepino[6,7,1-ij]quinoline;
1-fluoro-4,5,6,7,9,9a,10,11,12,13,14,14a-dodecahydrocyclohepta[c][1,4]diazepino[6,7,1-
ij]quinoline;
1-(trifluoromethyl)-4,5,6,7,9,9a,10,11,12,12a-decahydrocyclopenta[c][1,4]diazepino[6,7,-
ij]quinoline;
1-fluoro-2-methoxy-4,5,6,7,9,9a,10,11,12,12a-decahydrocyclopenta[c][1,4]diazepino[6,7,1-
ij]quinoline;
1-fluoro-2-methoxy-4,5,6,7,9,9a,10,11,12,13,14,14a-dodecahydrocyclo-
hepta[c][1,4]diazepino[6,7,1-ij]quinoline;
4,5,6,7,9,9a10,11,12,12a-decahydrocyclopenta[c][1,4]diazepino[6,7,1-ij]quinoline;
4,5,6,7,9,9a,10,11,12,13,14,14a-dodecahydrocyclohepta[c][1,4]diazepino[6,7,1-ij]quinoline;
(-)-4,5,6,7,9,9a10,11,12,12a-decahydrocyclopenta[c][1,4]diazepino[6,7,1-ij]quinoline;
(9aR,14aS)-4,5,6,7,9,9a,10,11,12,13,14,14a-dodecahydrocyclohepta[c][1,4]diazepino[6,7,1-
ij]quinoline;
(9aS,14aR)-4,5,6,7,9,9a,10,11,12,13,14,14a-dodecahydrocyclohepta[c][1,4]diazepino[6,7,1-
ij]quinoline;
4,5,6,7,9a,10,11,12,13,13a-decahydro-9H-[1,4]diazepino[6,7,1-de]phenanthridine;
1,2,3,4,9,10-hexahydro-8H-cyclopenta[b][1,4]diazepino[6,7,-hi]indole;
1,2,3,4,8,9,10,10a-octahydro-7bH-cyclopenta[b][1,4]diazepino[6,7,1-hi]indole;
(7bS,10aS)-1,2,3,4,8,9,10,10a-octahydro-7bH-cyclopenta[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
6-methyl-1,2,3,4,9,10-hexahydro-8H-cyclopenta[b][1,4]diazepino[6,7,1-hi]indole;
2S)-(rel-7bR,10aR)-2-methyl-1,2,3,4,8,9,10,10a-octahydro-7bH-cyclopenta[b]
[1,4]diazepino[6,7,1-hi]indole;
(2S)-(rel-7bR,10aR)-2-methyl-1,2,3,4,8,9,10,10a-octahydro-7bH-cyclopenta[b]
[1,4]diazepino[6,7,1-hi]indole;
(2S)-(rel-7bS,10aS)-2-methyl-1,2,3,4,8,9,10,10a-octahydro-7bH-cyclopenta[b]
[1,4]diazepino[6,7,1-hi]indole;
(2R)-(rel-7bR,10aR)-2-methyl-1,2,3,4,8,9,10,10a-octahydro-7bH-cyclopenta[b]
[1,4]diazepino[6,7,1-hi]indole;
(2R)-(rel-7bR,10aR)-2-methyl-1,2,3,4,8,9,10,10a-octahydro-7bH-cyclopenta[b
][1,4]diazepino[6,7,1-hi]indole;
(2R)-(rel-7bS,10aS)-2-methyl-1,2,3,4,8,9,10,10a-octahydro-7bH-cyclopenta[b]
[1,4]diazepino[6,7,1-hi]indole;
rel-(4S,7bS,10aS)-4-methyl-1,2,3,4,8,9,10,10a-octahydro-7bH-
cyclopenta[b][1,4]diazepino[6,7,1-hi]indole
rel-(4S,7bS,10aS)-4-methyl-1,2,3,4,8,9,10,10a-octahydro-7bH-cyclopenta[b]-
[1,4]diazepino[6,7,1-hi]indole;
rel-(4R,7bS,10aS)-4-methyl-1,2,3,4,8,9,10,10a-octahydro-7bH-
cyclopenta[b][1,4]diazepino[6,7,1-hi]indole;
9-methyl-1,2,3,4,8,9,10,10a-octahydro-7bH-cyclopenta[b][1,4]diazepino[6,7,1-hi]indole;
(7bR,9R,10aR)-9-methyl-1,2,3,4,8,9,10,10a-octahydro-7bH-cyclopenta[b][1,4]diazepino[6,7,1-
hi]indole;
9,9-dimethyl-1,2,3,4,8,9,10,10a-octahydro-7bH-cyclopenta[1,4]diazepino[6,7,1-hi]indole;
(7bR,10aR)-9,9-dimethyl-1,2,3,4,8,9,10,10a-octahydro-7bH-cyclopenta[b][1,4]diazepino[6,7,1-
hi]indole; and
(7bS,10aS)-9,9-dimethyl-1,2,3,4,8,9,10,10a-octahydro-7bH-cyclopenta[b][1,4]diazepino[6,7,1-
hi]indole;
or a pharmaceutically acceptable salt thereof. Another aspect of the present invention provides
the hydrochloride salt of each of the above compounds.

Also, it will be appreciated by those of ordinary skill in the art that reference to a compound herein is intended to include reference to any and all related forms such as polymorphs, hydrates, etc. Also, compounds may be provided as pro-drugs or other forms converted into the active agent during manufacture, processing, formulation, delivery, or in the body.

It will additionally be appreciated that the principles of the present invention apply all radiolabelled forms of compounds recited herein, including, for example, those where the radiolabels are selected from as ³H, ¹¹C, ¹⁴C, ¹⁸F, ¹²³I and ¹²⁵I. Such radiolabelled compounds are useful as research and diagnostic tools in metabolism pharmacokinetics studies and in binding assays in both animals and humans.

Compounds of formula I for use in accordance with the present invention may be obtained or produced according to any available means including methods described in detail in U.S. patent application Ser. No. 10/422,524, filed Apr. 24, 2003, and in U.S. provisional patent application Ser. No. 60/625,300, filed Nov. 5, 2004, the entirety of each of which is hereby incorporated herein by reference.

Without wishing to be bound by any particular theory, the present inventors note that compounds of formula I are highly specific agonists of the 5HT_{2C} receptor. Specifically, the present invention connects the observations that neurotransmitter 5-HT plays a major role in inhibition of nociceptive transmission and that various studies have demonstrated that at least 4 families of 5-HT receptors are present in pain processing pathways and include 5-HT1, 5-HT2, 5-HT3, and 5-HT4 (Doly et al., J Comp Neurol. 476(4):316-329, 2004; Ridet et al., J. Neurosc. Res 38(1):109-21, 1994). Furthermore, although exact mechanisms are poorly understood, it appears that 5-HT_{2C} receptors have an inhibitory role in neuropathic pain (Obata et al., Pain 108(1-2):163-9, 2004; Sasaki et al., Anesthesia & Analgesia, Baltimore, Md., 96(4):1072-1078, 2003; Obata et al., Brain Research 965(1-2): 114-20, 2003). According to the present invention, therefore, 5-HT_{2C} agonists may be effective in the treatment of diabetic neuropathy, post herpetic neuralgia, low back pain, phantom limb pain, visceral pain (chronic and acute), irritable bowel syndrome pain, irritable bowel disease pain, fibromyalgia and complex regional pain syndrome. Moreover, the present invention encompasses the recognition that the unique affinity and selectivity displayed by compounds of formula I can provide effective treatment of pain. Furthermore, the present invention recognizes that compounds of formula I may treat pain at lower doses and/or with fewer side effects than are observed with other available treatments.

2. Pharmaceutical Compositions

Compounds of formula I may be administered neat in order to treat pain in accordance with the present invention. More commonly, however, they are administered in the context of a pharmaceutical composition that, in addition to containing a pain treating effective amount of one or more compound of formula I, may include one or more ingredients known to those skilled in the art for formulating pharmaceutical compositions. Such ingredients include, for example, carriers (e.g., in solid or liquid form), flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Solid pharmaceutical compositions preferably contain one or more solid carriers, and optionally one or more other additives such as flavoring agents, lubricants, solubilizers, suspending agents, fillers, glidants, compression aids, binders or tablet-disintegrating agents or an encapsulating material. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes or ion exchange resins, or combinations thereof. In powder pharmaceutical compositions, the carrier is preferably a finely divided solid which is in admixture with the finely divided active ingredient. In tablets, the active ingredient is generally mixed with a carrier having the necessary compression properties in suitable proportions, and optionally, other additives, and compacted into the desired shape and size. Solid pharmaceutical compositions, such as powders and tablets, preferably contain up to 99% of the active ingredient.

Liquid pharmaceutical compositions preferably contain one or more compounds of formula I and one or more liquid carriers to form solutions, suspensions, emulsions, syrups, elixirs, or pressurized compositions. Pharmaceutically acceptable liquid carriers include, for example water, organic solvents, pharmaceutically acceptable oils or fat, or combinations thereof. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators, or combinations thereof. If the liquid formulation is intended for pediatric use, it is generally desirable to avoid inclusion of alcohol.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other pharmaceutically acceptable propellant.

Liquid pharmaceutical compositions which are sterile solutions or suspensions can be administered parenterally, for example by, intramuscular, intraperitoneal, epidural, intrathecal, intravenous or subcutaneous injection. Pharmaceutical compositions for oral or transmucosal administration may be either in liquid or solid composition form.

In certain embodiments of the present invention, the pharmaceutical composition, in addition to containing a compound of formula I may also contain therapeutically effective amounts of one or more other pain relieving agents and/or one or more other pharmaceutically active agents (see below for further discussion). Thus, the present invention also provides a pharmaceutical composition for treating pain comprising a pain treating effective amount of at least two different agents that each individually has pain treating activity, at least one agent being a compound of formula I. Those of ordinary skill in the art will appreciate that the amount of either agent required to provide a “pain treating effective amount” in such a combination may be different from the amount that is required to provide a pain treating effective amount of that agent alone. In certain embodiments, a lower amount of at least one of the pain treating agents is required in the combination than alone. In some embodiments of the invention, pain is treated using a combination of a compound of formula I and an opioid analgesic.

In some embodiments of the invention, pharmaceutical compositions are provided in unit dosage form, such as tablets or capsules. In such form, the composition is sub-divided in unit dose containing appropriate quantities of the active ingredient(s). The unit dosage forms can be packaged compositions, for example packeted powders, vials, ampoules, pre-filled syringes or sachets containing liquids. The unit dosage form can be, for example, a capsule or tablet itself, or it can be an appropriate number of any such compositions in package form.

Thus, the present invention also provides a pharmaceutical composition in unit dosage form for treating pain in a mammal that contains a pain treating effective unit dosage of at least one compound of formula I. As one skilled in the art will recognize, the preferred pain treating effective unit dosage will depend on for example the method of administration. A typical dosage of compounds of formula I often ranges from about 0.5 mg to about 500 mg, in some embodiments from about 1 mg or about 10 mg to about 500 mg.

The present invention also provides a therapeutic package for dispensing compounds of formula I to a mammal being treated for pain. In some embodiments, the therapeutic package contains one or more unit dosages of compounds of formula I, a container containing the one or more unit dosages, and labeling directing the use of the package for treating pain in a mammal. In certain embodiments, the unit dose is in tablet or capsule form. In some cases, each unit dosage is a pain treating effective amount.

3. Other Agents

Compounds of formula I may be administered alone in order to treat pain in accordance with the present invention, or may be combined with one or more other pharmaceutical agents. In some embodiments of the invention, the additional pharmaceutical agent(s) also have pain-relief activity. Alternatively or additionally, the additional agents may relieve one or more side effects associated with the pain-relieving agent(s), or may relieve one or more other symptoms or conditions associated with the pain or otherwise of concern to the individual suffering from or susceptible to pain.

Thus, in accordance with the present invention, the term “pain relieving agents” is used to refer to any agent that directly or indirectly treats pain or pain symptoms. Examples of indirect pain relieving agents include, for example, anti-inflammatory agents, such as anti-rheumatoid agents.

Where the present invention involves administration of two or more pharmaceutical agents, such as for example two or more pain relieving agents, the two or more agents may be administered simultaneously (such as individually at the same time, or together in a pharmaceutical composition), and/or successively with one another. In general, a compound of formula I and the other pharmaceutical agent(s) are administered in a manner so that both are present in the mammal body for a certain period of time to treat pain.

Also, the two or more pharmaceutical agents may be delivered via the same route of administration or by different routes. Desirable routes of administration may well depend upon the particular agent(s) chosen, many of which have recommended administration route(s) known to those skilled in the art. For example, opioids are generally administered by oral, intravenous, or intramuscular administration routes. Similarly, as is known in the art, doses of pharmaceutical agents in a composition may be affected by administration route. In general, pharmaceutical agents may be dosed and administered according to practices known to those skilled in the art such as those disclosed in references such as the Physicians' Desk Reference, 55 Edition, 2001, published by Medical Economics Co., Inc., Montvale, N.J.

Examples of pain relieving agents that may be administered with compounds of formula I in accordance with present invention include, but are not limited to, analgesics such as non-narcotic analgesics or narcotic analgesics; anti-inflammatory agents such as non-steroidal anti-inflammatory agents (NSAIDs), steroids or anti-rheumatic agents; migraine preparations such as beta adrenergic blocking agents, ergot derivatives, or isometheptene; tricyclic antidepressants such as amitryptyline, desipramine, or imipramine; anti-epileptics such as gabapentin, carbamazepine, topiramate, sodium valproate or phenyloin; a2 agonists; or selective serotonin reuptake inhibitors/selective norepinepherine uptake inhibitors, or combinations thereof.

One skilled in the art will recognize that some agents described herein act to relieve multiple conditions such as pain and inflammation, while other agents may just relieve one symptom such as pain. A specific example of an agent having multiple properties is aspirin, where aspirin is anti-inflammatory when given in high doses, but at lower doses is just an analgesic. The pain relieving agent may include any combination of the aforementioned agents, for example, the pain relieving agent may be a non-narcotic analgesic in combination with a narcotic analgesic.

Non-narcotic analgesics useful in the practice of the present invention include, for example, salicylates such as aspirin, ibuprofen (MOTRIN®, ADVIL®), ketoprofen (ORUDIS®), naproxen (NAPROSYN®), acetaminophen, indomethacin or combinations thereof. Examples of narcotic analgesic agents that may be used in combination with compounds of formula I include opioid analgesics such as fentenyl, sufentanil, morphine, hydromorphone, codeine, oxycodone, buprenorphine or pharmaceutically acceptable salts thereof or combinations thereof. Examples of anti-inflammatory agents that may be used in combination with compounds of formula I include but are not limited to aspirin; ibuprofen; ketoprofen; naproxen; etodolac (LODINE®); COX-2 inhibitors such as celecoxib (CELEBREX®), rofecoxib (VIOXX®), valdecoxib (BEXTRA®), parecoxib, etoricoxib (MK663), deracoxib, 2-(4-ethoxy-phenyl)-3-(4-methanesulfonyl-phenyl)-pyrazolo[1,5-b]pyridazine, 4-(2-oxo-3-phenyl-2,3-dihydrooxazol-4-yl)benzenesulfonamide, darbufelone, flosulide, 4-(4-cyclohexyl-2-methyl-5-oxazolyl)-2-fluorobenzenesulfonamide), meloxicam, nimesulide, 1-Methylsulfonyl-4-(1,1-dimethyl-4-(4-fluorophenyl)cyclopenta-2,4-dien-3-yl)benzene, 4-(1,5-Dihydro-6-fluoro-7-methoxy-3-(trifluoromethyl)-(2)-benzothiopyrano(4,3-c)pyrazol-1-yl)benzenesulfonamide, 4,4-dimethyl-2-phenyl-3-(4-methylsulfonyl)phenyl)cyclo-butenone, 4-Amino-N-(4-(2-fluoro-5-trifluoromethyl)-thiazol-2-yl)-benzene sulfonamide, 1-(7-tert-butyl-2,3-dihydro-3,3-dimethyl-5-benzo-furanyl)-4-cyclopropyl butan-1-one, or their physiologically acceptable salts, esters or solvates; sulindac (CLINORIL®); diclofenac (VOLTAREN®); piroxicam (FELDENE®); diflunisal (DOLOBID®), nabumetone (RELAFEN®), oxaprozin (DAYPRO®), indomethacin (INDOCIN®); or steroids such as PEDIAPED® prednisolone sodium phosphate oral solution, SOLU-MEDROL® methylprednisolone sodium succinate for injection, PRELONE® brand prednisolone syrup.

Further examples of anti-inflammatory agents that may be used for treating pain, for example associated with rheumatoid arthritis, in accordance with the present invention include naproxen, which is commercially available in the form of EC-NAPROSYN® delayed release tablets, NAPROSYN®, ANAPROX® and ANAPROX® DS tablets and NAPROSYN® suspension from Roche Labs, CELEBREX® brand of celecoxib tablets, VIOXX® brand of rofecoxib, CELESTONE® brand of betamethasone, CUPRAMINE® brand penicillamine capsules, DEPEN® brand titratable penicillamine tablets, DEPO-MEDROL brand of methylprednisolone acetate injectable suspension, ARAVA™ leflunomide tablets, AZULFIDIINE EN-tabs® brand of sulfasalazine delayed release tablets, FELDENE® brand piroxicam capsules, CATAFLAM® diclofenac potassium tablets, VOLTAREN® diclofenac sodium delayed release tablets, VOLTAREN®-XR diclofenac sodium extended release tablets, or ENBREL® etanerecept products.

Examples of yet other agents used to treat inflammations, especially rheumatoid arthritis, include immunosuppressants such as GENGRAF™ brand cyclosporine capsules, NEORAL® brand cyclosporine capsules or oral solution, or IMURAN® brand azathioprine tablets or IV injection; INDOCIN® brand indomethacin capsules, oral suspension or suppositories; PLAQUENIL® brand hydroxychloroquine sulfate; or REMICADE® infliximab recombinant for IV injection; or gold compounds such as auranofin or MYOCHRISYINE® gold sodium thiomalate injection.

The present invention provides pain treatments in which compounds of formula I are administered with one or more other pharmaceutical agents other than a pain-relieving agent. For example, in accordance with the present invention, compounds of formula I may be administered with one or more other pharmaceutical agents active in treating any other symptom or medical condition present in the mammal that is related or unrelated to the pain being experienced by the mammal. Examples of such pharmaceutical agents include, for example, anti-angiogenic agents, anti-neoplastic agents, anti-diabetic agents, anti-infective agents, or gastrointestinal agents, or combinations thereof.

A more complete list of pharmaceutically active agents, including pain relieving agents, can be found in the Physicians' Desk Reference, 55 Edition, 2001, published by Medical Economics Co., Inc., Montvale, N.J. Each of these agents may be administered in conjunction with one or more compounds of formula I according to the present invention. For most or all of these agents, recommended effective dosages and regimes are known in the art; many can be found in the above-referenced Physicians' Desk Reference, 55 Edition, 2001, published by Medical Economics Co., Inc., Montvale, N.J.

4. Uses

According to the present invention, compounds of formula I are useful for treating, or delaying the onset of, pain in mammals. By “treating”, as that term is used herein, it is meant partially or completely alleviating, inhibiting, ameliorating and/or relieving pain. For example, “treating” as used herein includes partially or completely alleviating, inhibiting or relieving pain for a period of time. “Treating” also includes completely ameliorating the pain. The term “delay the onset of” refers to a delay in the initiation of pain after a trigger. In some cases, the magnitude of the pain eventually suffered may also be reduced; in some instances, pain may be completely avoided.

Thus, in some embodiments of the present invention, compounds of formula I are administered after the onset of pain; in other embodiments, the compounds are administered prior to the onset of pain, for example after exposure to a stimulus that is expected or considered likely to induce pain.

According to the present invention, compounds of formula I may be used to treat any of a variety of different types of pains experienced by mammals, such as humans. For example, compounds of formula I may be used to treat treating acute pain (short duration) or chronic pain (regularly reoccurring or persistent), whether centralized or peripheral.

Examples of pain that can be acute or chronic and that can be treated in accordance with methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain, or headaches such as migraines or tension headaches, or combinations of these pains. One skilled in the art will recognize that these pains may overlap one another. For example, a pain caused by inflammation may also be visceral or musculoskeletal in nature.

In one embodiment of the present invention, one or more compounds of formula I is/are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromylagia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In some embodiments, compounds of formula I are used to treat chronic pain that is neuropathic pain, visceral pain, musculoskeletal pain, bony pain, headache, cancer pain or inflammatory pain or combinations thereof, in accordance with methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. Inventive treatment methods further include treatments in which the neuropathic pain is a condition secondary to metastatic infiltration, adiposis dolorosa, burns or central pain conditions related to thalamic conditions.

Neuropathic pains described above may also be, in some circumstances, classified as “painful small fiber neuropathies” such as idiopathic small-fiber painful sensory neuropathy, or “painful large fiber neuropathies” such as demylinating neuropathy or axonal neuropathy, or combinations thereof. Such neuropathies are described in more detail, for example, in the J. Mendell et al., N. Engl. J. Med. 2003, 348:1243-1255, which is hereby incorporated by reference in its entirety.

In another embodiment, compounds useful in the present invention may be administered to totally or partially inhibit a neuropathic pain condition from developing. For example, compounds of the present invention may be administered to a mammal who is at risk for developing a neuropathic pain condition such as a mammal who has contracted shingles or a mammal who is being treated for cancer.

In one embodiment, compounds useful in the present invention may be administered prior to or during a surgical procedure to partially or totally inhibit development of pain associated with the surgical procedure.

As mentioned previously, methods of the present invention may be used to treat pain that is somatic and/or visceral in nature. For example, somatic pain that can be treated in accordance with methods of the present invention includes pain associated with structural or soft tissue injury experienced during surgery, dental procedures, burns, or traumatic body injuries. Examples of visceral pain that can be treated in accordance with methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to methods of the present invention may also be related to conditions of hyperalgesia, allodynia, or both. Additionally, chronic pain to be treated in accordance with the present invention may be with or without peripheral or central sensitization.

The present invention also provides use of compounds of formula I to treat acute and/or chronic pains associated with female conditions, which may also be referred to as female-specific pain. Such types of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

According to the present invention, compounds of formula I can be administered in any of a variety of ways including for example by oral, intramuscular, intraperitoneal, epidural, intrathecal, intravenous, subcutaneous, intramucosal such as sublingual or intranasal, or transdermal administration. In a certain embodiments of the invention, compounds of formula I are administered orally, intramucosally or intravenously.

The present invention provides treatment methods in which compounds of formula I are administered in a pain treating effective amount to a mammal needing treatment for pain. As used herein “a pain treating effective amount” is at least the minimal amount of a compound of formula I, or a pharmaceutically acceptable salt form thereof, which reduces, alleviates, delays, and/or eliminates the pain in question.

To determine the pain treating effective amount of the compound to be administered in the treatment of pain in a particular circumstance, the physician may, for example, evaluate the effects of a given compound of formula I in the patient by incrementally increasing the dosage, for example from about 0.5 mg to about 1000 mg until the desired symptomatic relief level is achieved. The continuing dose regimen may then be modified to achieve the desired result. Similar techniques may be followed by determining the effective dose range for different administration routes.

### Exemplification

### EXAMPLE 1

Assessment of Effectiveness in Treatment of Pain

Compounds of formula I may be evaluated in accordance with the present invention to establish the extent of their effectiveness to treat pain, and may optionally be compared with other pain treatments.

A variety of methods have been established in the art to evaluate the effectiveness of compounds for relieving pain. See e.g., Bennett et al, Pain 33: 87-107, 1988; Chaplan et al, J. Neurosci. Methods 53:55-63, 1994; and Mosconi et al, Pain 64:37-57, 1996. Below is a specific description of one strategy that may be employed.

Procedure

Individually housed Spraque-Dawley rats are given free access to rat chow and water. A 12-h light/12-h dark cycle is put in effect (lights on from 6:00 am to 6:00 pm). Animal maintenance and research are conducted in accordance with the guidelines provided by the National Institutes of Health Committee on Laboratory Animal Resources. These subjects are used in the tests as set forth below.

Test Method 1: Prostaglandin E₂-induced thermal hypersensitivity.

The terminal 10 cm of the tail is placed into a thermos bottle containing water warmed to 38, 42, 46, 50, 54, or 58° C. The latency in seconds for the animal to remove the tail from the water is used as a measure of nociception. If the animal does not remove the tail within 20 sec, the experimenter removes the tail from the water and a maximum latency of 20 sec is recorded.

Following the assessment of baseline thermal sensitivity, thermal hypersensitivity is produced by a 50 μL injection of 0.1 mg prostaglandin E₂ (PGE₂) into the terminal 1 cm of the tail. Temperature-effect curves are generated before (baseline) and after (15, 30, 60, 90 and 120 min) the PGE₂ injection. Previous studies in other species (e.g., monkeys; Brandt et al., J. Pharmacol. Exper. Ther. 296:939, 2001) have demonstrated that PGE₂ produces a dose- and time-dependent thermal hypersensitivity that peaks 15 min after injection and dissipates after 2 hr.

Single compound studies. The ability of drugs to reverse PGE₂-induced thermal hypersensitivity is assessed using a single dose time-course procedure. Under this procedure, a single dose of the compound to be tested is administered intraperitoneally (IP), orally (PO) or intranasally (IN) 30 min before the injection of PGE₂. Tactile sensitivity is assessed 30 min after PGE₂ injection.

Combination compound studies. Combination studies with two or more potential pain treatment agents can be conducted. A minimally effective dose of a first agent, e.g., morphine is administered alone and in combination with ineffective doses of one or more compounds of formula I in the thermal warm-water tail withdrawal assay. Compounds are administered IP at the same time 30 min before testing.

Combination studies can also be conducted in the PGE₂-induced thermal hypersensitivity assay. For example, a dose of morphine that completely reverses thermal hypersensitivity (i.e., return to baseline) can be administered alone and in combination with doses of one or more compounds of formula I in the PGE₂-induced thermal warm-water tail withdrawal assay. Compounds are administered IP at the same time as PGE₂, which is administered 30 min before testing.

Test Method 1 Data Analysis—The temperature that produced a half-maximal increase in the tail-withdrawal latency (i.e., T₁₀) is calculated from each temperature-effect curve. The T₁₀ is determined by interpolation from a line drawn between the point above and the point below 10 sec on the temperature-effect curve. For these studies, thermal hypersensitivity is defined as a leftward shift in the temperature-effect curve and a decrease in the T₁₀ value. Reversal of thermal hypersensitivity is defined as a return to baseline of the temperature-effect curve and the T₁₀ value and is calculated according to the following equation:

$$\% MPE = \frac{( {T}_{10}^{drug + PGE 2} ) - ( {T}_{10}^{PGE 2} )}{( {T}_{10}^{baseline} ) - ( {T}_{10}^{PGE 2} )} \times 100$$

in which T₁₀^{drug+PGE2} is the T₁₀ after a drug in combination with PGE₂, T₁₀^{PGE2} is the T₁₀ after PGE₂ alone, and T₁₀^{baseline} is the T₁₀ under control conditions. A % MPE value of 100 indicates a complete return to the baseline thermal sensitivity observed without the PGE₂ injection. A value of greater than 100% indicates that the compound tested reduced thermal sensitivity more than the baseline thermal sensitivity without the PGE₂ injection.
Test Method 2: Chronic Constriction Injury

Rats are anesthetized with 3.5% halothane in O₂ at 1 L/min and maintained with 1.5% halothane in O₂ during surgery. A modified chronic sciatic nerve constriction injury (Mosconi & Kruger, 1996; Bennett & Xie, 1988) is produced by a cutaneous incision and a blunt dissection through the biceps femoris to expose the sciatic nerve. A PE 90 Polyethylene tubing (Intramedic, Clay Adams; Becton Dickinson Co.) cuff (2 mm length) is placed around the sciatic nerve at the level of the mid-thigh. The wound is closed in layers using 4-0 silk suture and wound clips. Testing is conducted 6-10 days after surgery.

Animals are placed in elevated wire cages and allowed 45-60 minutes to acclimate to the testing room. Baseline tactile sensitivity is assessed using a series of calibrated von Frey monofilaments (Stoelting; Wood Dale, Ill.) 0-3 days before surgery. Von Frey monofilaments are applied to the mid-plantar hind paw in sequential ascending or descending order, as necessary, to hover as closely as possible to the threshold of responses. The threshold is indicated by the lowest force that evoked a brisk withdrawal response to the stimuli. Thus, a withdrawal response leads to the presentation of the next lighter stimulus and the lack of a withdrawal response leads to the presentation of the next stronger stimulus. Rats with baseline thresholds <4 g force are excluded from the study. Approximately one week following CCI surgery, tactile sensitivities are reassessed and animals that exhibit motor deficiency (i.e. paw dragging) or failure to exhibit subsequent tactile hypersensitivity (threshold ≧10 g) are excluded from further testing. Under cumulative dosing conditions, compounds are administered IP every 30 minutes with the cumulative dose increasing in ½ log unit increments. Tactile hypersensitivity is assessed 20-30 minutes following each drug administration.

Test Method 2 Data Analysis. The 50% threshold values (in gm force) estimated by the Dixon non-parametric test (Chaplan et al, 1994) are calculated and fifteen-grams of force is used as the maximal force. Dose-effect curves are generated for each experimental condition for each rat. Individual tactile hypersensitivity threshold values are averaged to provide a mean (±1 SEM). Reversal of tactile hypersensitivity was defined as a return to baseline tactile sensitivity and was calculated according to the following equation:

$$\% Reversal = \frac{( 50 {\%}^{drug + CCI} ) - ( 50 {\%}^{CCI} )}{( 50 {\%}^{baseline} ) - ( 50 {\%}^{CCI} )} \times 100$$

in which 50%^{drug+CCI} is the 50% value after compound in animals approximately one week after CCI surgery, 50%^{CCI} is the 50% value approximately one week after CCI surgery alone, and 50% baseline is the 50% value before CCI surgery. Maximal effect of 100% reversal represents a return to the mean pre-operative threshold value for subjects in that experimental condition.
Test Method 3: Scheduled-Controlled Responding.

Rats are trained under a multiple-cycle procedure during experimental sessions conducted five days each week. Each training cycle consists of a 10-min pretreatment period followed by a 10-min response period. During the pretreatment period, stimulus lights are not illuminated, and responding has no scheduled consequences. During the response period, the left or right stimulus lights are illuminated (counterbalanced among subjects), the response lever is extended and subjects can respond under a fixed ratio 30 schedule of food presentation. Training sessions consist of 3 consecutive cycles. Testing sessions are identical to training sessions except that a single dose of drug is administered at the start of the first cycle.

Test Method 3 Data analysis. Operant response rates from individual animals are averaged for the three cycles during test sessions and are converted to percent of control response rates using the average rate from the previous training day as the control value (i.e., average of three cycles). Data are presented as the mean (±1 SEM) response rate as a percent of control. Thus, for example, a test value of 100% would indicate the response rate after administration of the compound to be tested is the same as the control response rate and there is no adverse effect of the compound tested.

### EXAMPLE 2

Assessment of Effectiveness in Chronic Neuropathic Pain Model

Compound:

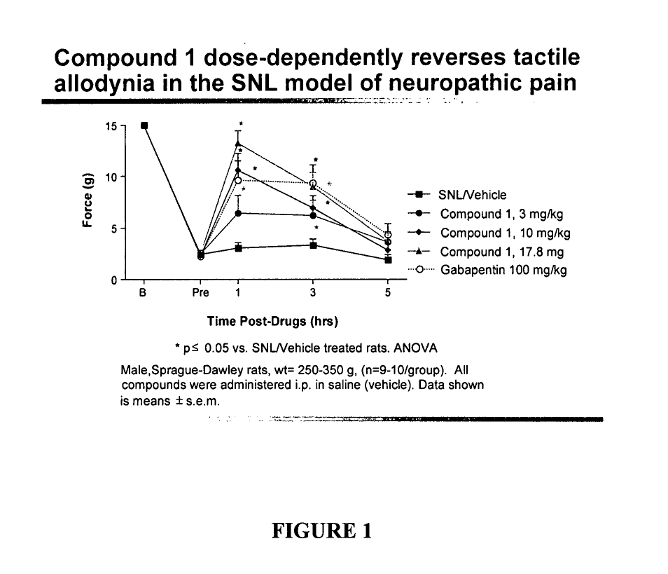

Compound 1, 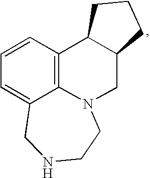
was obtained from Wyeth compound repository and gabapentin was purchased from Toronto Research Chemicals (Ontario, Canada) and were purchased from Sigma Chemical Company (St. Louis, Mo.). Compound 1 was dissolved in sterile saline and gabapentin was suspended in 2% Tween 80 in 0.5% methylcellulose and sterile water. All compounds were administered intraperitoneally (i.p.).
Subjects: Male Sprague-Dawley rats (125-150 g, Harlan; Indianapolis, Ind.) were individually housed on bedding. For all studies animals were maintained in climate-controlled rooms on a 12-hour light/dark cycle (lights on at 0630) with food and water available ad libitum.
Surgery: All surgical procedures were performed under 4% isoflurane/O₂ anesthesia, delivered via nose cone and maintained at 2.5% for the duration of the surgery.
L5 Spinal Nerve Ligation (SNL): Surgery was performed as previously described (Kim and Chung, XXX) with the exception that nerve injury was produced by tight ligation of the left L5 spinal nerve.
Assessment of Tactile Allodynia (Tactile Sensitivity): Tactile thresholds were assessed using a series of calibrated von Frey monofilaments (Stoelting; Wood Dale, Ill.). The threshold that produced a 50% likelihood of a withdrawal was determined using the up-down method, as previously described (Chaplan et al., 1994). Animals were placed in elevated wire cages and allowed 45-60 minutes to acclimate to the testing room. Von Frey monofilaments were applied to the mid-plantar left hind paw in sequential ascending or descending order, as necessary, to hover as closely as possible to the threshold of responses. The lowest force that evoked a brisk withdrawal response to the stimuli determined the pain threshold. Tactile thresholds were determined on the day prior to surgery and rats with baseline thresholds <10 g force were excluded from studies. Three weeks after SNL surgery tactile thresholds were reassessed and animals that failed to exhibit subsequent tactile allodynia (threshold ≧5 g) were excluded from further testing. Subjects were pseudo-randomly divided into test groups (n=8-10) so that average baseline and post-surgery sensitivities were similar among groups. Rats were administered Compound 1 (3, 10 or 17.8, i.p.) gabapentin (100 mg/kg, i.p., positive control) or vehicle and tactile thresholds were assessed up to 60, 180 and 300 minutes after dosing.
Analysis of Results: Statistical analysis was done using a repeated measures analysis of variance (ANOVA) using a customized SAS-excel application (SAS Institute, Cary, N.C.). Significant main effects were analyzed further by subsequent least significant difference analysis. The criterion for significant differences was p<0.05. Reversal of tactile allodynia was calculated according to the following equation:

$$\% Reversal = \frac{( 50 \% {threshold}^{drug + post surgery} ) - ( 50 \% {treshold}^{post surgery} )}{( 50 \% {threshold}^{pre surgery} ) - ( 50 \% {threshold}^{post surgery} )} \times 100$$

In which 50% threshold^{drug+post surgery} is the 50% threshold in g force after drug in nerve injured subjects, 50% threshold^{post surgery} is the 50% threshold in g force in nerve injured subjects, and 50% threshold^{pre surgery} is the 50% threshold in g force before nerve injury. Maximal effect of 100% reversal represents a return to the mean pre-operative threshold value for subjects in that experimental condition. See FIG. 1.

### EXAMPLE 3

Assessment of Effectiveness in Chronic Inflammatory Pain

Compound:

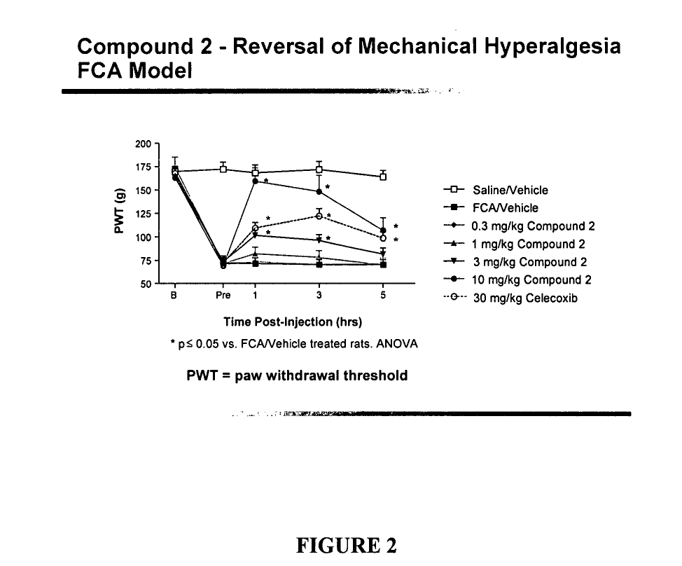

Compound 2, 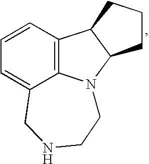
was obtained from Wyeth compound repository and celecoxib was purchased from Toronto Research Chemicals (Ontario, Canada). Compound 2 was dissolved in sterile saline and administered intraperitoneally (i.p.). Celecoxib was used as a positive control and was suspended in 2% Tween 80 in 0.5% methylcellulose and administered orally (p.o.).
Subjects: Male Sprague-Dawley rats (125-150 g, Harlan; Indianapolis, Ind.) were housed 3/cage on bedding and animals were maintained in climate-controlled rooms on a 12-hour light/dark cycle (lights on at 0630) with food and water available ad libitum.
Freund's complete adjuvant (FCA) of mechanical hyperalgesia: The hind paw withdrawal thresholds (PWTs) to a noxious mechanical stimulus were determined using an analgesimeter (model 7200; Ugo Basile). Cutoff was set at 250 g, and the endpoint taken was complete paw withdrawal. PWT was determined once for each rat at each time point (n=10/group). Baseline PWT was determined, and the rats were anesthetized with isofluorane (2% in oxygen) and received an intraplantar injection of 50% FCA (50 μl, diluted in saline) to the left hind paw. Twenty-four hours after FCA injection, pre-drug PWTs were measured, and the rats were administered vehicle or compound and assessed on PWTs 1, 3, 5, and 24 h post-drug administration.
Analysis of Results: Statistical analysis was done using a one way analysis of variance (ANOVA) using a customized SAS-excel application (SAS Institute, Cary, N.C.). Significant main effects are analyzed further by subsequent least significant difference analysis. The criterion for significant differences is p<0.05 from vehicle-treated FCA rats. Data is presented as percent reversal according to the following equation: percent reversal=[(post-dose threshold)-pre-dose threshold))/(baseline threshold-pre-dose threshold)]×100. See FIG. 2.

The entire disclosure of each patent, patent application, and publication cited or described in this document is hereby incorporated by reference.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

We claim:

1. A method of treating pain in a mammal, comprising administering to said mammal an effective amount of at least one compound of formula I: 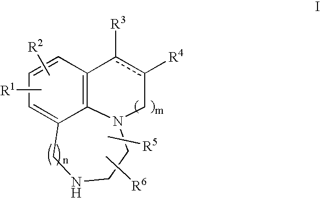
or a pharmaceutically acceptable salt thereof, wherein: [symbol]
designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R.

2. The method according to claim 1, wherein [symbol] designates a single bond.

3. The method according to claim 2, wherein:
R₁ is R, OR, halogen, cyano, or —C₁₋₃ perfluoroalkyl; and
R² is R, OR, halogen, cyano, or —C₁₋₃ perfluoroalkyl.

4. The method according to claim 3, wherein at least one of R¹ and R² is —OH.

5. The method according to claim 3, wherein R³ and R⁴ are taken together, with the carbon atoms to which they are bound, to form a saturated or unsaturated 5-8 membered ring, wherein said ring is optionally substituted with 1-3 groups independently selected from halogen, —R, or OR.

6. The method according to claim 1, wherein said compound is of formula I-a or I-b: 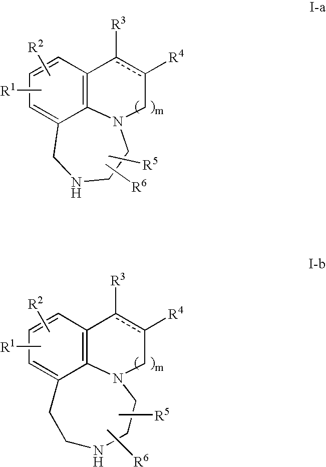
or a pharmaceutically acceptable salt thereof.

7. The method according to claim 1, wherein said compound is of formula I-c or I-d: 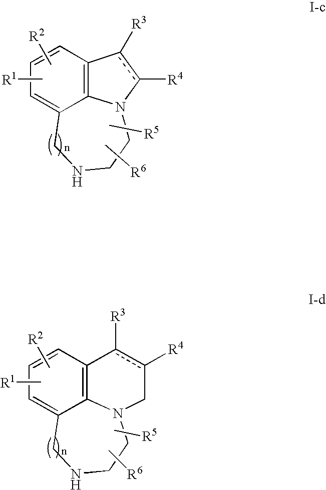
or a pharmaceutically acceptable salt thereof.

8. The method according to claim 7, wherein said compound is of formula II or III: 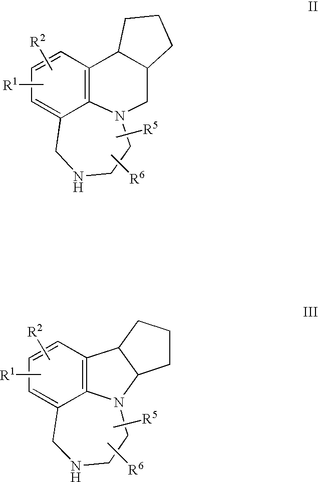
or a pharmaceutically acceptable salt thereof.

9. The method according to claim 1, wherein said compound is of formula I-e or I-f: 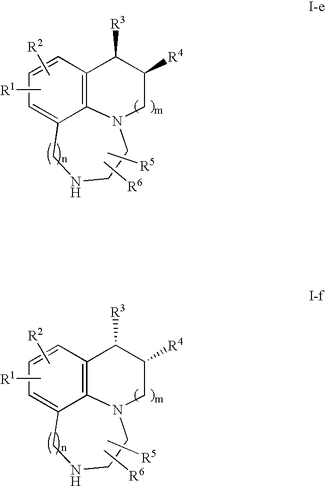
or a pharmaceutically acceptable salt thereof.

10. The method according to claim 9, wherein said compound is of formula IV or V: 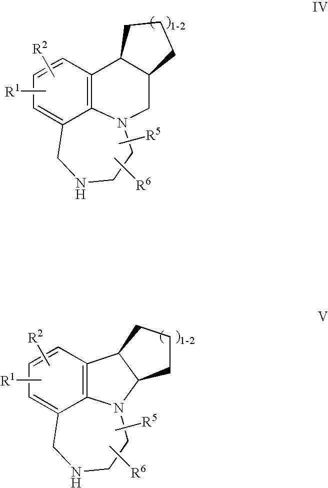
or a pharmaceutically acceptable salt thereof.

11. The method according to claim 1, wherein said compound is selected from:
2-bromo-4,5,6,7,9,9a,10,11,12,12a-decahydrocyclopenta[c][1,4]diazepino[6,7,1-ij]quinoline;
2-bromo-4,5,6,7,9,9a,10,11,12,13,14,14a-dodecahydrocyclohepta[c][1,4]diazepino[6,7,1-ij]quinoline;
2-chloro-4,5,6,7,9,9a,10,11,12,12a-decahydrocyclopenta[c][1,4]diazepino[6,7,1-ij]quinoline;
2-chloro-4,5,6,7,9,9a,10,11,12,13,14,14a-dodecahydrocyclohepta[c][1,4]diazepino[6,7,1—U]quinoline;
2-phenyl-4,5,6,7,9,9a,10,11,12,12a-decahydrocyclopenta[c][1,4]diazepino[6,7,1-ij]quinoline;
2-methoxy-4,5,6,7,9,9a,10,11,12,12a-decahydrocyclopenta[c][1,4]diazepino[6,7,1—U]quinoline;
1-fluoro-4,5,6,7,9,9a,10,11,12,12a-decahydrocyclopenta[c][1,4]diazepino[6,7,1-ij]quinoline;
1-fluoro-4,5,6,7,9,9a,10,11,12,13,14,14a-dodecahydrocyclohepta[c][1,4]diazepino[6,7,1—U]quinoline;
1-(trifluoromethyl)-4,5,6,7,9,9a,10,11,12,12a-decahydrocyclopenta[c][1,4]diazepino[6,7,1—U]quinoline;
1-fluoro-2-methoxy-4,5,6,7,9,9a,10,11,12,12a-decahydrocyclopenta[c][1,4]diazepino[6,7,1—U]quinoline;
1-fluoro-2-methoxy-4,5,6,7,9,9a,10,11,12,13,14,14a-dodecahydrocyclohepta[c][1,4]diazepino[6,7,1-ij]quinoline;
4,5,6,7,9,9a10,11,12,12a-decahydrocyclopenta[c][1,4]diazepino[6,7,1-ij]quinoline;
4,5,6,7,9,9a,10,11,12,13,14,14a-dodecahydrocyclohepta[c][1,4]diazepino[6,7,1-ij]quinoline;
(−)-4,5,6,7,9,9a10,11,12,12a-decahydrocyclopenta[c][1,4]diazepino[6,7,1—U]quinoline;
(9aR,14aS)-4,5,6,7,9,9a,10,11,12,13,14,14a-dodecahydrocyclohepta[c][1,4]diazepino[6,7,1-ij]quinoline; or
(9aS,14aR)-4,5,6,7,9,9a,10,11,12,13,14,14a-dodecahydrocyclohepta[c][1,4]diazepino[6,7,1-ij]quinoline;
4,5,6,7,9a,10,11,12,13,13a-decahydro-9H-[1,4]diazepino[6,7,1-de]phenanthridine;
1,2,3,4,9,10-hexahydro-8H-cyclopenta[b][1,4]diazepino[6,7,-hi]indole;
1,2,3,4,8,9,10,10a-octahydro-7bH-cyclopenta[b][1,4]diazepino[6,7,1-hi]indole;
(7bS,10aS)-1,2,3,4,8,9,10,10a-octahydro-7bH-cyclopenta[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
6-methyl-1,2,3,4,9,10-hexahydro-8H-cyclopenta[b][1,4]diazepino[6,7,1-hi]indole;
2S)-(rel-7bR,10aR)-2-methyl-1,2,3,4,8,9,10,10a-octahydro-7bH-cyclopenta[b][1,4]diazepino[6,7,1-hi]indole;
(2S)-(rel-7bR, 10aR)-2-methyl-1,2,3,4,8,9,10,10a-octahydro-7bH-cyclopenta[b][1,4]diazepino[6,7,1-hi]indole;
(2S)-(rel-7bS,10aS)-2-methyl-1,2,3,4,8,9,10,10a-octahydro-7bH-cyclopenta[b][1,4]diazepino[6,7,1-hi]indole;
(2R)-(rel-7bR,10aR)-2-methyl-1,2,3,4,8,9,10,10a-octahydro-7° b.H-cyclopenta[b][1,4]diazepino[6,7,1-hi]indole;
(2R)-(rel-7bR,10aR)-2-methyl-1,2,3,4,8,9,10,10a-octahydro-7bH-cyclopenta[b ][1,4]diazepino[6,7,1-hi]indole;
(2R)-(rel-7bS,10aS)-2-methyl-1,2,3,4,8,9,10,10a-octahydro-7bH-cyclopenta[b][1,4]diazepino[6,7,1-hi]indole;
rel-(4S,7bS,10aS)-4-methyl-1,2,3,4,8,9,10,10a-octahydro-7bH-cyclopenta[b][1,4]diazepino[6,7,1-hi]indole
rel-(4S,7bS,10aS)-4-methyl-1,2,3,4,8,9,10,10a-octahydro-7bH-cyclopenta[b]-[1,4]diazepino[6,7,1-hi]indole;
rel-(4R,7bS,10aS)-4-methyl-1,2,3,4,8,9,10,10a-octahydro-7bH-cyclopenta[b][1,4]diazepino[6,7,1-hi]indole;
9-methyl-1,2,3,4,8,9,10,10a-octahydro-7bH-cyclopenta[b][1,4]diazepino[6,7,1-hi]indole;
(7bR,9R,10aR)-9-methyl-1,2,3,4,8,9,10,10a-octahydro-7bH-cyclopenta[b][1,4]diazepino[6,7,1-hi]indole;
9,9-dimethyl-1,2,3,4,8,9,10,10a-octahydro-7bH-cyclopenta[1,4]diazepino[6,7,1-hi]indole;
(7bR,10aR)-9,9-dimethyl-1,2,3,4,8,9,10,10a-octahydro-7bH-cyclopenta[b][1,4]diazepino[6,7,1-hi]indole; and
(7bS,10aS)-9,9-dimethyl-1,2,3,4,8,9,10,10a-octahydro-7bH-cyclopenta[b][1,4]diazepino[6,7,1-hi]indole;
or a pharmaceutically acceptable salt thereof.

12. The method of claim 11, wherein said compound is the hydrochloride salt.

13. The method of claim 1, wherein the pain is acute pain or chronic pain.

14. The method of claim 15, wherein the pain is inflammatory pain, musculoskeletal pain, bony pain, lumbosacral pain, neck or upper back pain, visceral pain, somatic pain, neuropathic pain, cancer pain, pain caused by injury or surgery, or headache pain, or combinations thereof.

15. The method of claim 13, wherein the pain is chronic pain.

16. The method of claim 15, wherein the chronic pain is associated with allodynia, hyperalgesia, or both.

17. The method of claim 15, wherein the chronic pain is neuropathic pain; cancer pain; visceral pain; musculoskeletal pain; bony pain; headache pain; or pain associated with infections, sickle cell anemia, autoimmune disorders, multiple sclerosis, or inflammation, or combinations thereof.

18. The method of claim 1, wherein the pain comprises neuropathic pain.

19. The method of claim 18, wherein the neuropathic pain is associated with diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, phantom limb pain, reflex sympathetic dystrophy, postthoracotomy pain, cancer, chemical injury, toxins, nutritional deficiencies, or viral or bacterial infections, or combinations thereof.

20. The method of claim 1, further comprising administering a pharmaceutically effective amount of at least one pain relieving agent.

21. The method of claim 20, wherein the pain relieving agent comprises one or more analgesics; anti-inflammatory agents; migraine preparations; tricyclic antidepressants; anti-epileptics; α₂ agonists; or selective serotonin reuptake inhibitors/selective norepinepherine uptake inhibitors; or combinations thereof.

22. The method of claim 21, wherein the pain relieving agent comprises an opioid analgesic.
